(19) United States
(12) Patent Application Publication
(10) Pub. No.: US 2016/0338834 A1
(43) Pub. Date: Nov. 24, 2016

(54) MEDICAL SIPHON

(71) Applicant: Cook Medical Technologies LLC, Bloomington, IN (US)

(72) Inventor: John Eckberg, Bloomington, IN (US)

(73) Assignee: Cook Medical Technologies LLC, Bloomington, IN (US)

(21) Appl. No.: 15/158,436

(22) Filed: May 18, 2016

Related U.S. Application Data
(60) Provisional application No. 62/164,956, filed on May 21, 2015.

(51) Int. Cl. A61F 2/24; A61N 1/36; A61F 2/06

(52) U.S. Cl. CPC A61F 2/2475; A61F 2/06; A61N 1/36042; A61F 2/2418; A61F 2/2424

(57) ABSTRACT

Medical siphons and methods of pumping fluids are provided. A medical siphon may include a valve configured to be implanted in a body vessel, a sphincter disposed below the valve and wrapping around a wall of the body vessel, and a pacemaker disposed on the sphincter. The siphon may also include a component configured to communicate with the pacemaker, such as a blood pressure monitor or a flow probe.

### BACKGROUND

1. Field of the Invention

The present disclosure generally relates to medical devices. More particularly, the disclosure relates to medical siphons that may be used in connection with transporting fluids in the body.

2. Description of the Related Art

Chronic venous insufficiency (CVI) of the lower extremities is a common condition that is considered a serious public health and socioeconomic problem. In the United States, approximately two million workdays are lost each year, and over 2 million new cases of venous thrombosis are recorded each year. About 800,000 new cases of venous insufficiency syndrome will also be recorded annually. Ambulatory care costs of about $2,000, per patient, per month, contribute to the estimated U.S. cost of $16,000,000 per month for the treatment of venous stasis ulcers related to CVI.

It is estimated that greater than 3% of the Medicare population is afflicted by a degree of CVI manifested as non-healing ulcers. Studies have indicated that about 40% of seriously affected individuals cannot work or even leave the house except to obtain medical care. It is estimated that 0.2% of the U.S. work force is afflicted with CVI.

CVI arises from long duration venous hypertension caused by valvular insufficiency and/or venous obstruction secondary to venous thrombosis. Other primary causes of CVI include varicosities of long duration, venous hypoplasia and arteriovenous fistula. The signs and symptoms of CVI have been used to classify the degree of severity of the disease and reporting standards have been published. Studies demonstrate that deterioration of venous hemodynamic status correlates with disease severity. Venous reflux, measured by ultrasound studies, is the method of choice of initial evaluation of patients with pain and/or swelling in the lower extremities. In most serious cases of CVI, venous stasis ulcers are indicative of incompetent venous valves in all systems, including superficial, common, deep and communicating veins. This global involvement affects at least 30% of all cases. Standard principles of treatment are directed at elimination of venous reflux. Based on this observation, therapeutic intervention is best determined by evaluating the extent of valvula incompetence, and the anatomical distribution of reflux. Valvular incompetence, a major component of venous hypertension, is present in about 60% of patients with a clinical diagnosis of CVI.

Endovascular valve replacement is a concept that involves percutaneous insertion of the prosthetic device under fluoroscopic guidance. The device can be advanced to the desired intravascular location using guide wires and catheters. Deployment at a selected site can be accomplished to correct valvular incompetence. Percutaneous placement of a new valve apparatus provides a less invasive solution compared to surgical transposition or open repair of a valve. The prevalence of CVI and the magnitude of its impact demand development of an effective therapy.

### BRIEF SUMMARY

In one embodiment, the present disclosure relates to a medical siphon comprising a valve configured to be implanted in a body vessel, a sphincter disposed below the valve and wrapping around a wall of the body vessel, and a pacemaker disposed on the sphincter. In some embodiments, the sphincter is a porcine cardiac sphincter that has been decellularized and repopulated with stem cells.

In another embodiment, the disclosure relates to a method of pumping a bodily fluid comprising implanting a valve in a body vessel and wrapping a sphincter around an outer wall of the body vessel. The sphincter is placed distally adjacent the valve. The method also includes the step of disposing a pacemaker on the sphincter, wherein the pacemaker detects a pulse from a heart and stimulates the sphincter with an electrical impulse, thereby causing the sphincter to contract and push the bodily fluid through the valve. In some embodiments, the sphincter is surgically placed around the outer wall of the body vessel with vascularization occurring via surgical connection to appropriate vessels.

In an additional embodiment, the disclosure provides a method of pumping a bodily fluid comprising implanting a valve in a body vessel and wrapping a sphincter around an outer wall of the body vessel. The sphincter is placed distally adjacent the valve. The method also includes the steps of disposing a pacemaker on the sphincter and providing a component configured to communicate with the pacemaker, wherein the component configured to communicate with the pacemaker detects a pulse from a heart and sends an electrical signal to the pacemaker, wherein the pacemaker stimulates the sphincter with an electrical impulse, thereby causing the sphincter to contract and push the bodily fluid through the valve.

The foregoing has outlined rather broadly the features and technical advantages of the present disclosure in order that the detailed description that follows may be better understood. Additional features and advantages of the disclosure will be described hereinafter that form the subject of the claims of this application. It should be appreciated by those skilled in the art that the conception and the specific embodiments disclosed may be readily utilized as a basis for modifying or designing other embodiments for carrying out the same purposes of the present disclosure. It should also be realized by those skilled in the art that such equivalent embodiments do not depart from the spirit and scope of the disclosure as set forth in the appended claims.

### BRIEF DESCRIPTION OF THE SEVERAL VIEWS OF THE DRAWINGS

A detailed description of the invention is hereafter described with specific reference being made to the drawings in which:

FIG. 1 shows an example of a ball-and-socket valve;

FIG. 2 shows an additional embodiment of a ball-and-socket valve;

FIG. 3 shows an aspect of the presently disclosed siphon implanted in the body of a patient; and

FIG. 4 shows an embodiment of a leaf-flap/venous valve.

### DETAILED DESCRIPTION

Various embodiments are described below with reference to the drawings in which like elements generally are referred to by like numerals. The relationship and functioning of the various elements of the embodiments may better be understood by reference to the following detailed description. However, embodiments are not limited to those illustrated in the drawings. It should be understood that the drawings are not necessarily to scale, and in certain instances details may have been omitted that are not necessary for an understanding of embodiments disclosed herein, such as conventional fabrication and assembly.

In accordance with the present disclosure, the term “implantable” refers to an ability of a medical device to be positioned at a location within a body, such as within a body vessel. Furthermore, the terms “implantation” and “implanted” refer to the positioning of a medical device at a location within a body, such as within a body vessel.

The term “biocompatible” refers to a material that is substantially non-toxic in the in vivo environment of its intended use, and that is not substantially rejected by the patient's physiological system (i.e., is non-antigenic). This can be gauged by the ability of a material to pass the biocompatibility tests set forth in International Standards Organization (ISO) Standard No. 10993 and/or the U.S. Pharmacopeia (USP) 23 and/or the U.S. Food and Drug Administration (FDA) blue book memorandum No. G95-1, entitled “Use of International Standard ISO-10993, Biological Evaluation of Medical Devices Part-1: Evaluation and Testing.” Typically, these tests measure a material's toxicity, infectivity, pyrogenicity, irritation potential, reactivity, hemolytic activity, carcinogenicity and/or immunogenicity. A biocompatible structure or material, when introduced into a majority of patients, will not cause an undesirably adverse, long-lived or escalating biological reaction or response, and is distinguished from a mild, transient inflammation which typically accompanies surgery or implantation of foreign objects into a living organism.

The term “body vessel” means any passageway or lumen that conducts fluid, including, but not limited to, blood vessels, such as veins or arteries.

The term “antegrade fluid flow” refers to the flow of fluid, such as blood, in a primary direction of normal movement within a body vessel. For example, in veins, antegrade fluid flow proceeds primarily toward the heart. The term “retrograde fluid flow” refers to fluid flow in a direction opposite the primary (antegrade) direction of fluid flow. For example, retrograde flow in a vein is primarily directed away from the heart.

A “venous valve-related condition” is any condition presenting symptoms that can be diagnostically associated with improper function of one or more venous valves.

Finally, in accordance with the present disclosure, the terms “proximal” and “distal” describe longitudinal directions in opposing axial ends of a medical device, such as an implantable valve, and components thereof.

The term “proximal” is used in its conventional sense to refer to the end of the device (or component) that is closest to the clinician conducting the implantation of the medical device within a body vessel. The term “distal” is used in its conventional sense to refer to the end of the device (or component) that is initially farther from the clinician implanting the device.

Many vessels in animals transport fluids from one body location to another in a substantially unidirectional manner along the length of the vessel. Native valves within the heart and veins function to regulate blood flow within these body vessels. For example, heart valves direct the flow of blood into and out of the heart and to other organs, while venous valves direct the flow of blood toward the heart. Body vessels, such as veins, transport blood to the heart, while arteries carry blood away from the heart.

In many mammals, small semilunar valves, known as “venous valves” (valvulae vienosa), are found within the extremity veins. Such venous valves function as one-way check valves to maintain the flow of venous return blood toward the heart, while preventing blood from back-flowing away from the heart. Heart valves open and close 60 to 150 times per minute with pressures of up to about 250 mm Hg. Venous valves typically remain open with minimal forward flow and close with flow reversal. Reverse venous flow may develop intermittent pressures of about 150 mm Hg. Venous valves are particularly important in the veins of the lower extremities, as venous blood returning from the lower extremities is required to move against a long hydrostatic column, especially when the subject in a standing or upright position.

Venous valves are typically bicuspid valves positioned at varying intervals within veins to permit substantially unidirectional blood to flow toward the heart. These natural venous valves open to permit the flow of fluid in the desired direction, and close upon a change in pressure, such as a transition from systole to diastole. When blood flows through the vein, the pressure forces the valve leaflets apart as they flex in the direction of blood flow and move towards the inside wall of the vessel, creating an opening therebetween for blood flow. The venous valve leaflets, however, do not normally bend in the opposite direction, and therefore return to a closed position to restrict or prevent blood flow in the opposite, i.e. retrograde, direction after the pressure is relieved. The venous valve leaflet structures, when functioning properly, extend radially inwardly toward one another such that the tips contact each other to restrict backflow of blood. In the present disclosure, the terms “leaf-flap valve” and “venous valve” may be used interchangeably.

Venous valves, especially those in the upper leg, perform an important function. When a person rises from a seated to a standing position, arterial blood pressure increases instantaneously to insure adequate perfusion to the brain and other critical organs. In the legs and arms, the transit time of this increased arterial pressure is delayed, resulting in a temporary drop in venous pressure. The venous pressure in the feet of someone walking is of the order of 25 mmHg (3.3 kPa), whereas, in the feet of an individual standing absolutely still, it is of the order of 90 mmHg (12 kPa). A properly functioning venous valve detects drops in pressure, and the resulting change of direction of blood flow, and closes to prevent blood from pooling. For example, in the legs, to maintain blood volume in the heart and head. The valves reopen and the system returns to normal forward flow when the reflected arterial pressure again appears in the venous circulation. Compromised valves, however, would allow reverse blood flow and pooling.

Occasionally, congenital defects or injury to valves within a body vessel can result in an undesirable amount of retrograde fluid flow across a valve therein, and compromise the unidirectional flow of fluid across the valve. In an embodiment, the present disclosure is directed to a siphon comprising a valve that may be implanted in a body vessel containing an improperly functioning valve.

In some embodiments, the presently disclosed siphon can be used to treat lymphedema. The siphon can address major build-ups of fluid leading from the lymph system and lymph nodes. The siphon could be implanted in the thoracic duct near the subclavian vein and the “clenching” pressure of nearby muscles triggered by a pacemaker could enable fluids to be forcibly drained into the vein from the lymphatic system. Additional areas in the lymphatic system that could receive a siphon are the lymphatic ducts, which drain into one of the two subclavian veins, near the junction with the internal jugular veins.

The presently disclosed siphon includes implantable components, such as a valve, that can be inserted within various body vessels, such as veins, to modify the direction of fluid flow. Any known minimally invasive techniques and/or catheter delivery systems may be used to implant the components of the presently disclosed siphon. Various percutaneous methods of implanting medical devices within the body using intraluminal transcatheter delivery systems can also be used for implantation of any component of the siphon. Any component of the presently disclosed siphon can be introduced to a point of treatment within a body vessel using a delivery catheter device passed through the vasculature communicating between a remote introductory location and the implantation site, and released from the delivery catheter device at the point of treatment within the body vessel. Any component of the siphon may be deployed in a body vessel at a point of treatment and the delivery device subsequently withdrawn from the vessel, while the component(s) of the siphon is retained within the vessel and can restore blood flow at the target site, for example, in the leg of a patient. An implanted valve of the siphon can improve the function of native valves by blocking or reducing retrograde fluid flow. Alternatively, a valve of the siphon can be implanted to replace the function of damaged or absent native valves within the body.

The presently disclosed siphon is capable of desirably modifying fluid flow within a body vessel while maintaining fluid flow across the siphon. Fluid flow modification can include permitting fluid to flow in a first direction with a lower resistance than in the opposite, retrograde direction, thereby enhancing, improving, or replacing the function of one-way venous valves. The presently disclosed siphon may be used in connection with any bodily fluid, such as blood, lymph fluid, ascites fluid, etc.

The present disclosure also provides methods of treating various medical conditions, such as venous valve-related conditions, by modifying fluid flow through a body vessel. The methods may include the endoluminal implantation of any component of the siphon for regulating fluid flow within a body vessel, such as a vein, in a manner providing a greater resistance to fluid flow through the body vessel in a retrograde direction than in an antegrade direction. In some embodiments, the siphon may be configured to restrict the rate of fluid flow within the body vessel by about 0.5 to about 30% when passing through the valve of the siphon.

The siphon is preferably configured to reduce the rate of fluid flow through a body vessel in a flow direction-dependent manner, such as by reducing the rate of fluid flow in a first direction less than in a second direction. For example, the siphon may be adapted to reduce blood flow in an antegrade direction less than fluid flow in a retrograde direction. In some embodiments, the siphon does not reduce flow in the antegrade direction. The siphon may be configured to provide a greater resistance to fluid flow across the valve of the siphon in the retrograde direction than in the antegrade reduction. For example, a fluid flow passing across the valve of the siphon in a retrograde direction may be reduced by about 0.1-100% more than the same rate and pressure of fluid flow in the opposite, antegrade direction.

The presently disclosed siphon may have various configurations and components that, when working together, enable a fluid, such as blood, to flow against gravity to the heart. In some embodiments, the siphon comprises one or more components selected from the group consisting of one or more valves, a sphincter, a bladder, a pacemaker, and/or a component configured to communicate with the pacemaker, such as flow probe or blood pressure monitor.

The valve may be implanted in a body vessel, such as a vein. In accordance with the present disclosure, it is to be understood that the term “valve” not only includes a single valve, but may also include multiple valves, such as a first valve connected to a second valve by a bladder. In some embodiments, the valve is implanted in a posterior tibial vein or femoral vein in the upper leg. In certain embodiments, the valve is implanted at a location in the superficial venous system, such as in a saphenous vein in a leg. Alternatively, the valve may be implanted in the deep venous system, such as in a femoral vein and/or a popliteal vein. In other embodiments, the valve may be implanted in a body vessel of the leg within the gastrocnemius, peroneus, and/or tibialis anterior muscles. In some embodiments, more than one valve can be implanted in the same body vessel or one or more valves may be implanted in a first body vessel and a second valve (or a second valve, a third valve, a fourth valve, etc.) may be implanted in a second body vessel, e.g., one or more valves in a vein of one leg and one or more valves in a vein of the other leg. Each valve may also be associated with other components of the siphon, such as a sphincter, a pacemaker, a bladder, a flow probe, and/or any other component disclosed in connection with the siphon systems described herein.

Any biocompatible valve may be used in connection with the presently disclosed siphon. In some embodiments, the valve comprises a flexible material with sufficient rigidity, similar to cartilage, which does not cause occlusions or endothelial responses in the body. For example, the valve, or any component thereof, may comprise porcine tissue. The porcine tissue may be decellularized and repopulated, as is described more fully below. In some embodiments, the valve is a leaf-flap valve or a ball-and-socket valve. In certain embodiments, the ball-and socket valve may be 3-D printed. In any embodiment, the valve may comprise radiopaque markers, such as one or more gold markers, to assist with placement of the valve in a body vessel. Additionally, in any embodiment, the proximal end of the valve may comprise a filter, such as the Celect® Filter available from Cook Medical.

In certain embodiments, the valve may be a leaf-flap valve that is sewn to a frame, such as a Nitinol frame, and comprises a plurality of leafs. In some embodiments, the leafs may comprise porcine material, such as porcine tissue, and may be sewn, bioglued, or otherwise anchored into place. In other embodiments, for example, the valve may be a leaf-flap valve comprising a porcine material that is sewn to a framework, such as a Nitinol framework, and then depopulated with a detergent-enzynmatic treatment before it is repopulated in vitro with human-induced pluripotent stem cells. Any known leaf-flap valves may be used, such as those disclosed in U.S. Patent Application Publication No. 2008/0051879 titled “Methods of treating venous valve related conditions with a flow-modifying implantable medical device”, the disclosure of which is incorporated into the present application in its entirety.

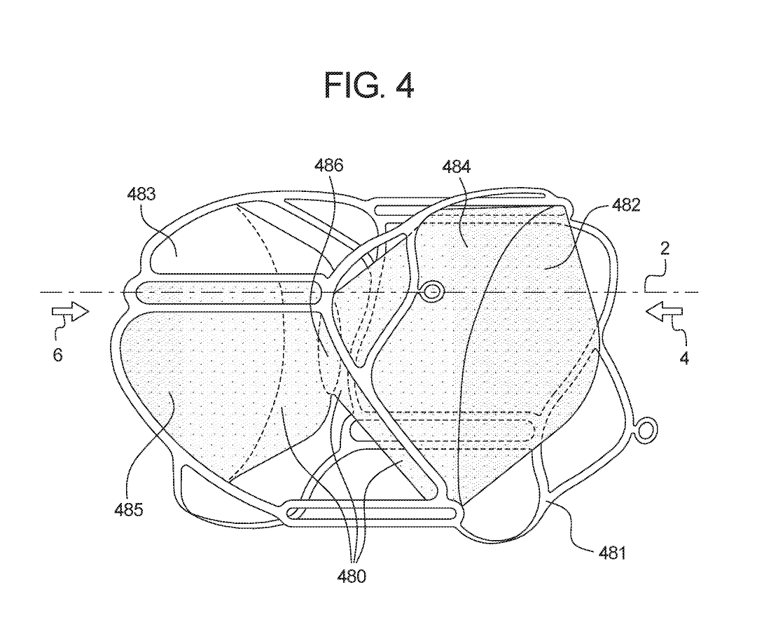

One example of this type of valve is depicted in FIG. 4. The leaf-flap valve of FIG. 4 comprises a forming film (480) formed from, for example, a biocompatible polyurethane attached to an implantable frame (481) disposed around the forming film (480). The forming film (480) forms a bi-directional fluid flow restricting channel extending along the longitudinal axis (2) from an inlet (482) to an outlet (483). The forming film (480) defines an antegrade flow receiving surface (484) and a retrograde flow receiving surface (485) joined at an orifice (486). The forming material (480) is rigid enough to direct fluid into and through the orifice (486) from either longitudinal direction. By configuring the retrograde flow receiving surface (485) and the antegrade flow receiving surface (484) differently, fluid flow passing though the orifice (486) is preferably reduced more in the retrograde direction (6) than in the antegrade direction (4). The forming film can be attached to a suitable support frame (481) configured to maintain the fluid flow restricting channel in a desired geometry. The support frame can optionally provide a stenting function to the medical device (i.e., exert a radially outward force on the interior wall of a vessel in which the medical device is implanted). By including a support frame that exerts such an outward radial force, the device can provide both a stenting and a flow-modifying function at a point of treatment within a body vessel.

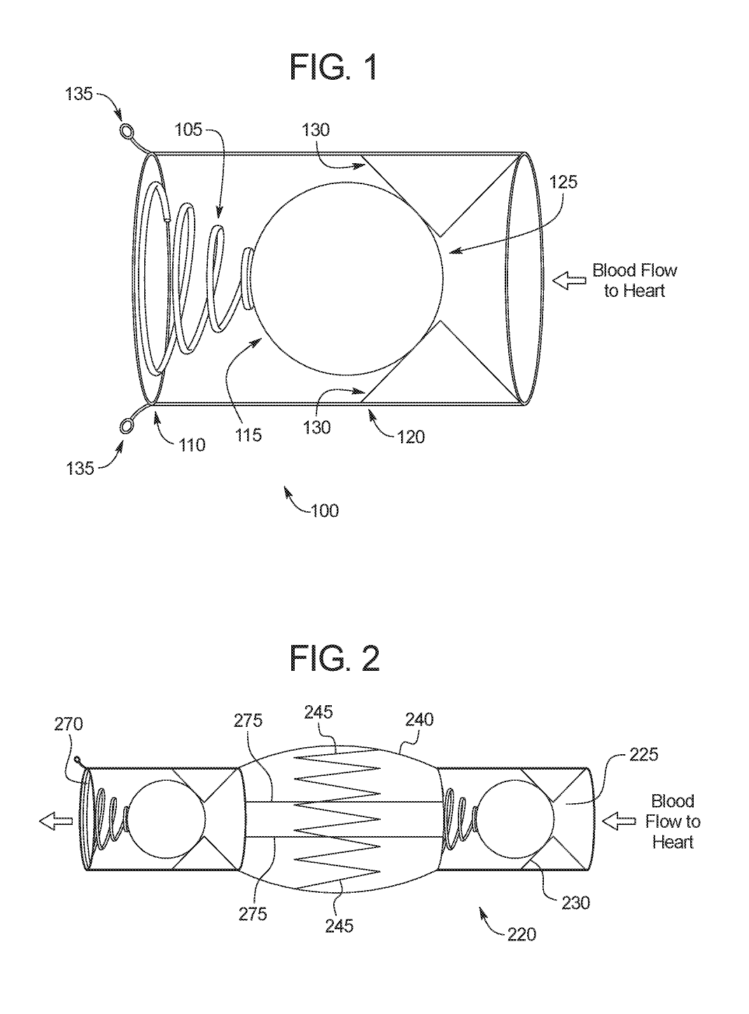

In other exemplary embodiments, such as seen in FIG. 1, the valve (100) may be 3-D printed to from a ball-and-socket valve, also known as a floor valve, and the material used may be any biocompatible polymer, such as casein. In some embodiments, a spring (105) may be disposed between, and connected or anchored to, at least one of, the proximal end of the valve (110) and the proximal side (115) of the ball. The spring may be made from any biocompatible material, such as Nitinol. As blood flows through the valve housing (120), the ball moves proximally and the spring is compressed. When the blood pressure is reduced, the spring urges the ball in the distal direction, thereby closing the opening (125) of the valve. In some embodiments, the springs may be absent. In these embodiments, the pressure of the force of the compression can push the lower ball valve closed and upon relaxation, the siphoning effect and/or gravity of the fluid would close the top valve.

In some embodiments, the surface (130) of the valve contacting a distal portion of the ball may be coated with a material, such as urethane, to improve the seal between the distal portion of the ball and the opening in the valve. The valve housing (120) may further comprise radiopaque markers (135), such as gold markers, useful for placement and identification of valve location.

To prevent migration of the valve, the valve housing (120) may comprise one or more barbs and/or small protrusions, at its proximal, mid, and/or distal sections to anchor the valve in place within the vessel. Barbs are commonly known to assist in anchoring stents within a body passage and this known barb technology can be incorporated into the housing (120) of any of the presently disclosed valves. In addition, in some embodiments, the outer diameter of the valve may be slightly (such as about 1 mm) larger than the inner diameter of the body vessel. As such, the valve will provide a radial force to the inner vessel wall to help hold the valve in place.

While in some embodiments the body vessels are arteries, in other embodiments, the body vessels may not have concentric circumferences and cylindrical shapes like an artery. Since the valve(s) (and the bladder) will be conforming to the anatomic vasculature, the interior of the valve device may have concentric volume but the exterior or the portion that adjoins the walls of the body vessel may not be in that same shape. That is, a CT scan can yield a 3-D image that may have distinct configurations and therefore, the 3-D printed valve will also have a surface that will conform to the vessel walls. Therefore, the valve can lock into place once it is percutaneously implanted and the radial force can keep it from migrating.

The valve may comprise a flow orifice having an outer diameter that is substantially the same as, or slightly larger than, the inner diameter of the body vessel. The valve may comprise any biocompatible materials, such as medical grades of PVC and polyethylene, PEEK, casein, polycarbonate, polysulfone, polypropylene, polyurethane, and any combination thereof. Advantageously, when implanted, the valve will not create intimal hyperplasia. Additionally, the valve may be custom printed based on CT scans of individuals who are going to receive the implanted valve.

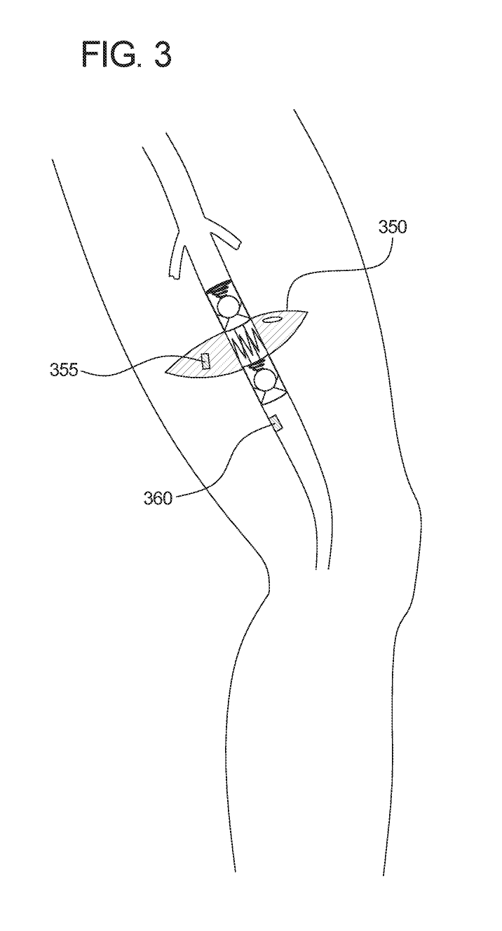

In any embodiment, such as that shown in FIG. 2, the valve may also comprise a bladder (240). In some embodiments, the bladder may comprise a non-porous, woven chamber. In other embodiments, the bladder may comprise rubber or urethane, for example. The bladder (240) may be a flexible conduit that provides a fluid-impermeable connection between a distal end valve and a proximal end valve. That is, as shown in FIG. 2, any of the implantable devices described herein may include two valves connected to each other by a bladder. The bladder may be attached to each valve by any known means, such as using an adhesive.

In some embodiments, the rubber or urethane chamber of the bladder may comprise patterns or indentations in its wall to assist with flexibility. For example, the wall of the bladder may comprise a series of indentations, or a waffle-like pattern, or it may have a series of foldable ridges like an accordion. If woven, the wall of the bladder may comprise any materials that can be woven, such as a fabric, yarn, thread, etc. The bladder (240) may have substantially the same circumference as the inner valve lumen (245).

As seen in FIG. 2, the bladder (240) may also balloon slightly outwards such that its mid-portion comprises a larger outer diameter than its proximal and distal ends. In some embodiments, the bladder (240) may be reinforced with one or more wires (245), such as Nitinol wires, which can contract and expand, along with the bladder, and may provide a radial force to assist the bladder (240) in expanding after a contraction. In some embodiments, the wire may be wrapped around the circumference of the inner-bladder wall and stiched or sewed to the inner-wall to prevent migration. The shape of the wire is not critical and, in some embodiments, the wire may comprise a cylindrical, spiral configuration.

The first and second valves shown in FIG. 2 comprise spring-biased ball-and-socket valves. With respect to the proximal (or first) ball valve, the proximal end of the spring may be anchored into the proximal end of the valve using any known techniques, such as adhesives, bioglue, stitching, etc. The distal end of the spring may optionally be anchored to the ball. The proximal end of the spring associated with the distal (or second) ball valve may be anchored to the distal end of the bladder using any known techniques, such as adhesives, bioglue, stitching, etc. The distal end of the spring may optionally be anchored to the ball.

In some embodiments, the inner wall of the valve may comprise one or more ridges/rims (270) to prevent the ball from flowing out of the valve in case of siphon malfunction. For example, in the valve depicted in FIG. 2, the proximal end of the valve may comprise a rim (270) having a smaller inner diameter than the diameter of the ball. At the distal end, the inner valve surface (230) may comprise an opening (225) that has a smaller diameter than the diameter of the ball. In some embodiments, the bladder (240) may comprise one or more reinforcing spurs (275) to prevent movement of the ball valves during compression. The reinforcing spurs (275) may contain Nitinol, for example, and, in some embodiments, may be attached to, or formed within, the wall of the bladder. In addition to the previously described characteristics for preventing migration of the valve within the body vessel, the valve housing (220) may also comprise a corrugated pattern, protrusions, ridges, and the like, on its outer surface to prevent migration.

When the implantable device comprises a first (proximal) valve separated from a second (distal) valve by a bladder, the sphincter may be wrapped around the bladder, on the outside of the body vessel, as can be seen in FIG. 3. Compression/contraction of the sphincter causes the bladder to compress/contract. In turn, this causes any blood or fluid in the bladder to push the proximal ball valve open, thereby allowing the blood or fluid to flow out of the valve. After contraction, the sphincter and bladder return to their radially-expanded configuration, which causes the proximal ball valve to close since a vacuum is created. In turn, blood is allowed to flow into the distal end of the valve, thereby pushing open the distal end ball valve and entering the bladder.

While the valve is implanted inside the body vessel, the sphincter may be placed below the valve, distally adjacent the valve, around the outside of the body vessel. For example, if the valve was implanted in a vein in a leg of a patient, the sphincter would be placed distally adjacent the valve, such that it would be closer to the foot of the patient than the valve. The sphincter wraps around the outer vessel wall. Further, if the implantable device comprises a first valve and a second valve separated from one another by a bladder, the sphincter may be placed below the first (proximal) valve, distally adjacent the valve, around the bladder on the outside of the body vessel.

While any sphincter may be used, in some embodiments, the sphincter may come from, for example, the duodenum of an animal, such as a pig. In other embodiments, a human sphincter may be used, such as a sphincter from a cadaver, or, in some embodiments, skeletal muscle fibers may be used, such as slow-twitch muscles and/or fast-twitch muscles. The sphincter may be wrapped around the body vessel and the separated ends may be sewed together with sutures to reconnect the sphincter. In some embodiments, the suture line may be covered with a reinforcing layer in order to ensure compression of the muscle during electrical signaling. In some embodiments, the reinforcing material may comprise the submucosal layer of a pig intestine.

In some embodiments, the sphincter may be a reconstituted porcine tissue sphincter that has been decellularized through enzymes and repopulated with autologous/mesenchymal stem cells. The decellularizing and repopulating processes (for the sphincter and the valve) can be carried out by one having ordinary skill in the art. The decellularization may occur through a detergent-enzymatic treatment, for example. Repopulating may occur with human induced pluripotent stem cell-derived progenitor cells, for example. The seeded, multipotential progenitor cells proliferate into cardiomyocytes, smooth muscle cells, and endothelial cells to reconstruct the sphincter. After perfusion is complete and placement occurs surgically, contractions are stimulated, for example, by electrical signals from a pacemaker implanted surgically into the reconstituted sphincter. The sphincter may be wrapped around pertinent veins or body vessels holding the percutaneously placed valve(s).

In certain embodiments, the sphincter may be disposed on, and surround, a sleeve. The sleeve may be wrapped around the body vessel. The sleeve may comprise a woven material that is reinforced with one or more metallic bands, such as Nitinol bands, that keep/urge the sleeve open after compression. The sleeve may be sutured to the vessel and the suture line may be reinforced with a reinforcing layer as described above.

As mentioned, in some embodiments, the sphincter may comprise a pacemaker. For example, a pacemaker may be disposed on a surface of the sphincter or the pacemaker may be implanted within the sphincter. Some pacemakers comprise a threaded, screw-type end, and thus may be screwed into the sphincter. Any pacemaker known in the medical arts may be used. The pacemaker may be used to stimulate the sphincter with electrical impulses. The pacemaker may be aligned with (in electronic communication with) the component configured to communicate with the pacemaker, if such a component is present. If the pacemaker can detect pulse, then a component configured to communicate with the pacemaker is unnecessary.

In one embodiment, the component configured to communicate with the pacemaker is a flow probe, such as a Doppler flow probe, which is a diagnostic instrument that emits an ultrasonic beam into the body. The Doppler flow probe may comprise a monitor and a sensor. It can estimate blood flow through blood vessels by bouncing high-frequency sound waves (ultrasound) off circulating red blood cells. Any known Doppler may be used as the flow probe. For example, the flow probe may comprise a Doppler DP-M350 or a Cook-Swartz Doppler, both commercially available from Cook Medical. In some embodiments, the target body vessel may comprise the sensor of the Doppler flow probe and the monitor may, for example, be worn by the patient around his or her ankle, finger, wrist, etc. In certain embodiments, the sensor of the flow probe may be disposed on an outer wall of the vessel or sutured around the vessel, below the valve and sphincter. In other embodiments, the sensor may be external and worn on the patient's leg, for example.

The sensor has the capability of sensing or detecting pulse or vascular pressure from the heart. When detected, the sensor may send a wireless electrical signal to the monitor (or some other type of controller, such as a cellular phone) and the monitor may send a wireless electrical signal to the pacemaker, causing it to stimulate the sphincter and cause contraction thereof.

Since the component configured to communicate with the pacemaker and the pacemaker are in communication, when a pulse from the heart is registered (detected) by the component configured to communicate with the pacemaker, the pacemaker is triggered, which causes the sphincter to contract, thereby pushing blood through the valve. If the valve is in a body vessel in the leg, when the sphincter contracts, the blood is pushed up into the valve, towards the heart. As noted above, more than one valve may be placed in the body vessel or different body vessels may comprise one or more valves.

In other embodiments, the siphon may not comprise a component configured to communicate with the pacemaker. In such embodiments, the pacemaker is configured such that it can detect pulse or vascular pressure from the heartbeat. If vascular blood pressure can be detected by the pacemaker, then a component configured to communicate with the pacemaker is not necessary. Additionally, instead of comprising a flow probe, the component configured to communicate with the pacemaker may comprise a blood pressure monitor. In some embodiments, the blood pressure monitor may be wearable in the vicinity of the sphincter. The wearable blood pressure monitor may be configured to send a wireless electrical signal to the pacemaker when it detects a pulse from the heart, thereby causing the pacemaker to stimulate the sphincter. Alternatively, the blood pressure monitor may send a wireless electrical signal to a controller, cellular telephone, etc., and the controller device may then send a wireless electrical signal to the pacemaker.

Various methods of treatment are contemplated by the present disclosure. These methods may include altering fluid flow within a body vessel, for example, to treat a venous valve-related condition. In particular, methods of altering fluid flow in a directionally-dependent manner are provided that may include the step of implanting a valve within a body vessel. The methods of treatment may include the step of delivering a valve to a body vessel, which may be a vein or other blood vessel in communication with the venous system, and implanting the valve in the body vessel. The valve can be configured to provide a fluid flow restriction channel that comprises an orifice that is substantially constant during changes in fluid flow pressure and/or direction. In some embodiments, the diameter of the orifice does not substantially change or close to prevent fluid flow in either antegrade or retrograde directions. In certain embodiments, fluid flow is reduced in one or both directions or eliminated in one direction.

In some embodiments, the valve (or any component of the siphon) is implanted percutaneously to a point of treatment in a body vessel using any suitable delivery device, including delivery catheters dilators, sheaths, and/or other suitable endoluminal devices. Alternatively, the valve (or any component of the siphon) can be placed in body vessels by any suitable technique, including percutaneous delivery, as well as surgical placement. FIG. 3 shows one example of various siphon system components implanted in the femoral artery. For example, the implantable valve (300) comprises a first and second valve, where the first/proximal valve is shown proximally adjacent to a sphincter (350). A pacemaker (355) is disposed on the sphincter (350) and the sphincter (350) is proximally adjacent to a sensor (360) of a flow probe. As noted above, the sphincter (350) may be disposed on (wrapped around) a sleeve. The sleeve is an optional component but may be wrapped around the body vessel for placement of the sphincter (350) thereon.

Multiple siphons may be provided, each comprising a valve (or multiple valves), and the valves can be inserted upstream (with respect to blood flow) and/or downstream of one or more venous valve leaflets. When a venous valve related condition is manifested by failure of valves within the upper (e.g. saphenous) and/or lower (e.g. popliteal) portions of the leg, the valve of the siphon may be implanted within a blood vessel lower (or higher) on the leg than the failed valve and, in some embodiments, a second valve may be implanted within a blood vessel higher (or lower) on the leg than the first valve.

All of the devices, components, and methods disclosed and claimed herein can be made and executed without undue experimentation in light of the present disclosure. While this invention may be embodied in many different forms, there are described in detail herein specific preferred embodiments of the invention. The present disclosure is an exemplification of the principles of the invention and is not intended to limit the invention to the particular embodiments illustrated. In addition, unless expressly stated to the contrary, use of the term “a” is intended to include “at least one” or “one or more.” For example, “a valve” is intended to include “at least one valve” or “one or more valves.”

Any ranges given either in absolute terms or in approximate terms are intended to encompass both, and any definitions used herein are intended to be clarifying and not limiting. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all sub-ranges (including all fractional and whole values) subsumed therein.

Furthermore, the invention encompasses any and all possible combinations of some or all of the various embodiments described herein. It should also be understood that various changes and modifications to the presently preferred embodiments described herein will be apparent to those skilled in the art. Such changes and modifications can be made without departing from the spirit and scope of the invention and without diminishing its intended advantages. It is therefore intended that such changes and modifications be covered by the appended claims.

What is claimed is:

1. A medical siphon comprising:
a proximal valve configured to be implanted in a body vessel;
a distal valve configured to be implanted in the body vessel, wherein the proximal valve and the distal valve are connected by a bladder;
a sphincter configured to be wrapped around a wall of the body vessel at a location between the proximal valve and the distal valve; and
a pacemaker disposed on the sphincter.

2. The medical siphon of claim 1, further comprising a component configured to communicate with the pacemaker.

3. The medical siphon of claim 2, wherein the component configured to communicate with the pacemaker is a flow probe comprising a sensor and a monitor.

4. The medical siphon of claim 3, wherein the sensor is disposed below the distal valve on the body vessel.

5. The medical siphon of claim 1, wherein the proximal valve and distal valve comprise porcine tissue.

6. The medical siphon of claim 5, wherein the porcine tissue has been decellularized by a detergent-enzymatic treatment and repopulated with human induced pluripotent stem cell-derived progenitor cells.

7. The medical siphon of claim 1, wherein the proximal valve and distal valve comprise a leaf-flap valve or a ball-and-socket valve.

8. The medical siphon of claim 1, wherein the sphincter comprises porcine tissue.

9. The medical siphon of claim 8, wherein the tissue has been decellularized by a detergent-enzymatic treatment and repopulated with human induced pluripotent stem cell-derived progenitor cells.

10. The medical siphon of claim 1, wherein the pacemaker is configured to detect vascular blood pressure.

11. The medical siphon of claim 7, wherein the ball and socket valve comprises a rim.

12. A method of pumping a bodily fluid comprising:
implanting a device in a body vessel, the device comprising a proximal valve connected to a distal valve by a bladder;
wrapping a sphincter around an outer wall of the body vessel, wherein the sphincter is placed at a location between the proximal valve and the distal valve;
disposing a pacemaker on the sphincter, wherein the pacemaker detects a pulse from a heart and stimulates the sphincter with an electrical impulse, thereby causing the sphincter to contract and push the bodily fluid through the proximal valve.

13. The method of claim 12, wherein the body vessel is selected from the group consisting of a vein, a lymphatic duct, and any combination thereof.

14. The method of claim 12, wherein at least one of the proximal valve and the distal valve comprises porcine tissue, wherein the tissue has been decellularized with a detergent-enzynmatic treatment and repopulated in vitro with human-induced pluripotent stem cells.

15. The method of claim 12, wherein the bodily fluid is selected from the group consisting of blood, ascites fluid, lymph fluid, and any combination thereof.

16. A method of pumping a bodily fluid comprising:
implanting a device in a body vessel, the device comprising a proximal valve connected to a distal valve by a bladder;
wrapping a sphincter around an outer wall of the body vessel, wherein the sphincter is placed at a location between the proximal valve and the distal valve;
disposing a pacemaker on the sphincter; and
providing a component configured to communicate with the pacemaker, wherein the component configured to communicate with the pacemaker detects a pulse from a heart and sends an electrical signal to the pacemaker, and wherein the pacemaker stimulates the sphincter with an electrical impulse, thereby causing the sphincter to contract and push the bodily fluid through the proximal valve.

17. The method of claim 16, wherein the component configured to communicate with the pacemaker is a flow probe or a blood pressure monitor.

18. The method of claim 17, wherein the flow probe comprises a sensor and a monitor, wherein the sensor is disposed on the body vessel, below the sphincter.

19. The method of claim 18, wherein the sensor detects the pulse from the heart, sends an electrical signal to the monitor, and the monitor sends an electrical signal to the pacemaker, thereby causing the pacemaker to stimulate the sphincter.

20. The method of claim 16, wherein the body vessel is selected from the group consisting of a vein, a lymphatic duct, and any combination thereof.
